(19) United States
(12) Patent Application Publication
(10) Pub. No.: US 2023/0027591 A1
(43) Pub. Date: Jan. 26, 2023

(54) METHOD AND APPARATUS FOR PASSIVE KNOWLEDGE ACQUISITION IN A DISTRIBUTED SYSTEM

(71) Applicant: SCALAMED PTY LTD, Bondi Junction, New South Wales (AU)

(72) Inventors: Alon Novy, Bondi Junction, New South Wales (AU); Tal Rapke, Bondi Junction, New South Wales (AU)

(21) Appl. No.: 17/756,956

(22) PCT Filed: Dec. 3, 2020
(86) PCT No.: PCT/AU20/51316

§ 371 Date: Jun. 6, 2022

(30) Foreign Application Priority Data
Dec. 6, 2019 (AU) 2019904624

(51) Int. Cl. G16H 80/00; G16H 10/60

(52) U.S. Cl. CPC G16H 80/00; G16H 10/60

(57) ABSTRACT

Disclosed herein are a method and system for delivering contextually relevant information from disparate information sources. The system includes: a set of responders, each responder being associated with at least one source of information; a situation generator for parsing an agent data set received from an agent to identify an actionable situation; and an intervention controller. The intervention controller: sends a notification of the actionable situation to a selected set of the responders; receives an intervention recommendation from at least one responder, the intervention recommendation being contextually relevant information for the identified actionable situation; determines whether the intervention recommendation is valid; and forwards the intervention recommendation to an intervention performer, when the intervention recommendation is valid.

### RELATED APPLICATION

The present application is related to Australian Provisional Patent Application No. 2019904624 titled “Method and apparatus for passive knowledge acquisition in a distributed system” and filed on 6 Dec. 2019 in the name of ScalaMed Pty Ltd, the entire content of which is incorporated by reference as if fully set forth herein.

### TECHNICAL FIELD

The present disclosure relates to a computer-based information system and associated method. In particular, the present disclosure relates to an information system for providing contextual information to a user, such as providing personalised health care guidance to a user.

### BACKGROUND

There exist today many sources of medical information, for example drug databases, medical records systems, discharge documents issued by hospitals, medical journals, medical web sites and blog posts, genomic databases, and alternative therapies, as well as brochures published by manufacturers of pharmaceutical products. Patients themselves may also be sources of medical information, either through their experiences or symptoms, or a combination thereof. Similarly, medical practitioners may also be sources of medical information through their knowledge and experience. Collectively, these sources of medical information make up a large body of medical knowledge.

In many instances, a particular person may be the only source of some medical information. For example, a patient may have been prescribed a medication, but whether that patient has taken the medication as prescribed may in many cases be known only to that patient. The patient may also be the only source of information relating to the effects of the medication, including any side effects.

An individual may, at one time, require access to one part of that medical knowledge and, at another time, require access to another part of that medical knowledge. For example, a particular patient, having just been prescribed a new medication, would benefit from knowing that this new medication has known, dangerous side-effects when taken together with another medication that particular patient is already taking. Such potential drug-drug interactions (DDIs) can be extremely dangerous and even life-threatening. Further, knowing that a prescribed medication has known side-effects may comfort a patient or associated carer that any experience of the known side-effects is normal or expected, and may alleviate concerns.

According to another example, a care co-ordinator of a medical practice may be interested to know which of her patients are not properly following or adhering to the treatments prescribed by the clinic, since this would enable the care coordinator to follow up pro-actively with the non-adherent patients to help prevent adverse health outcomes.

Unfortunately, because the body of medical knowledge is distributed among so many sources of medical information, for an individual to discover all medical information that is of relevance to the individual at any given moment would require having access to every source of medical information and competence for conducting an interrogation of those sources. As will be appreciated by those skilled in the art, no-one individual is likely to have access to all relevant sources of information, let alone at a given time, and in many cases the individual is furthermore unlikely to be competent at interrogating and properly assessing the information in the sources that are available to said individual.

There are at present initiatives to enable and simplify the exchange of medical information between different sources of information. Such initiatives include the creation of generally accepted standards for electronic data formats, e.g., Health Level Seven (HL7), and protocols for interoperability between software systems, e.g., Smart on FHIR.

Although such initiatives are a positive development, these approaches suffer from a number of serious limitations, including:
a. That any medical information that includes Private Personal Health Information (PPHI), also known as Private Health Information (PHI), relating to an individual (i.e., information that is deemed by laws or custom to be private to the individual referred to in the information) cannot be easily exchanged without compromising the privacy of an individual;
b. That setting up interoperability and/or the digital exchange of medical information between two sources of medical information requires a substantial degree of mutual trust between the parties operating the respective sources of medical information, as well as technical expertise. Therefore, such initiatives will proceed slowly and, in many cases, will likely not proceed at all;
c. That some of the medical information may be considered confidential or proprietary to the party that operates the source of that information and therefore the party will be unlikely to agree to share or pool the medical information with the operators of other sources of medical information, particularly where the operators are each other's competitors; and
d. That many individuals do not have the time, discipline, or know-how to conduct routine searches of the body of medical knowledge, or do not even know what questions to ask or where to go. Consequently, even medical information that is technically available to such individuals is likely to remain undiscovered by those individuals.

Thus, a need exists to provide an improved method and system for passive knowledge acquisition in a distributed system.

A further need exists to provide a method and system whereby a given individual may passively acquire personally relevant medical information as and when that information is relevant to the individual in a way that does not require the disclosure of restricted medical information to individuals who have no right to the restricted medical information and who are not properly authorised to receive the restricted medical information, restricted medical information being medical information that is not in the public domain for example because it is private, confidential or proprietary.

### SUMMARY

The present disclosure relates to a method and system for passive knowledge acquisition in a distributed system.

A first aspect of the present disclosure provides a system for delivering contextually relevant information, comprising:
a set of responders, each responder associated with at least one source of information;
a situation generator for parsing an agent data set received from an agent to identify an actionable situation;
an intervention controller for:
   sending a notification of said actionable situation to a selected set of said responders;
   receiving an intervention recommendation from at least one responder, the intervention recommendation being contextually relevant information for the identified actionable situation;
   determining whether the intervention recommendation is valid; and
   forwarding the intervention recommendation to an intervention performer, when said intervention recommendation is valid.

In some embodiments, the system relates to a medical practice, wherein the agent data set is an electronic medical record (EMR) relating to a patient, the actionable situation is a health situation, and each responder is a health situation responder associated with at least one source of medical information. The source of medical information may be a stored repository of data, such as relating to diseases, pharmaceuticals, anatomy, diagnoses, and the like, or may be access to one or more professionals able to advise on a received health situation, or a combination thereof.

A second aspect of the present disclosure provides a method for passively acquiring knowledge distributed among at least one source of information, comprising the steps of:
receiving at an intervention controller an agent data set associated with an agent;
analysing, by a situation generator associated with the intervention controller, the agent data set to identify an actionable situation;
notifying, by the intervention controller, the actionable situation to a set of at least one responder, wherein each responder has access to at least one source of information relating to the actionable situation;
receiving, at the intervention controller, an intervention recommendation from at least one of said responders;
forwarding, by said intervention controller, said intervention recommendation to an intervention performer for execution.

According to another aspect, the present disclosure provides an apparatus for implementing any one of the aforementioned methods.

According to another aspect, the present disclosure provides a computer program product including a computer readable medium having recorded thereon a computer program that when executed on a processor of a computer implements any one of the methods described above.

Other aspects of the present disclosure are also provided.

### BRIEF DESCRIPTION OF THE DRAWINGS

One or more embodiments of the present disclosure will now be described by way of specific example(s) with reference to the accompanying drawings, in which:

FIG. 1 is a schematic block diagram representation of a system that includes a general purpose computer on which one or more embodiments of the present disclosure may be practised;

FIG. 2 is a schematic representation of an object model depicting some of the principal components according to one embodiment of the present disclosure;

FIG. 3 shows a simplified flowchart for registering a Health Situation Responder;

FIG. 4 shows a simplified flowchart for generating a Reference Persona according to one embodiment of the disclosure;

FIG. 5 depicts, in simplified form, a sequence diagram illustrating the process executed in response to a generated Health Situation according to one embodiment of the present disclosure;

FIG. 6 depicts, in simplified form, an exemplary user interface for performing an Intervention;

FIG. 7 is a sequence diagram illustrating some of the method invocations involved in the generation and processing of a Health Situation;

FIG. 8 is a schematic representation illustrating components of a system in accordance with the present disclosure;

FIG. 9 is a schematic representation illustrating a sample set of principal data entities and various relationships, when a system in accordance with the present disclosure is implemented in relation to a healthcare environment; and

FIG. 10 is a schematic block diagram representation of a system for passively acquiring knowledge from at least one source of information.

Method steps or features in the accompanying drawings that have the same reference numerals are to be considered to have the same function(s) or operation(s), unless the contrary intention is expressed or implied.

### DETAILED DESCRIPTION

The present disclosure provides a method and system for implementing a method for passively acquiring knowledge distributed among at least one source of information, such as medical information. In the context of this specification, the term “knowledge” is to be broadly construed as referring to contextually relevant information. In one application, the method and system enable a patient to acquire contextually relevant information from disparate sources. The disparate sources may include, for example, but are not limited to, stored data repositories, such as libraries of documents, one or more professionals or otherwise qualified practitioners, or a combination thereof.

A system for delivering contextually relevant information is provided, wherein the system includes a set of responders, a situation generator for parsing an agent data set received from an agent to identify an actionable situation; and an intervention controller. Each responder is associated with at least one source of information. Thus, the set of responders provides access to a set of disparate sources of information. The set of responders may optionally be registered in a responder register accessible by the intervention controller.

The intervention controller controls the flow of information within the system. The intervention controller may be implemented, for example, by a computer server coupled to a communications network. The intervention controller sends a notification of the actionable situation to a selected set of the responders. By default, the selected set of responders may be all responders registered in a responder register. Alternatively, the selected set of responders may be determined based on attributes associated with the respective responders and/or attributes associated with the actionable situation.

The intervention controller receives an intervention recommendation from at least one responder, the intervention recommendation being contextually relevant information for the identified actionable situation. The intervention controller determines whether the intervention recommendation is valid and, when the intervention recommendation is valid, forwards the intervention recommendation to an intervention performer. Validity of the intervention recommendation is not restricted to whether the intervention recommendation is appropriate for the actionable situation, but may also be based on capabilities of the responder, past performance of the responder that provided the intervention recommendation, whether the responder satisfies criteria established by the agent data set or agent, or any combination thereof.

In some embodiments, the agent data set is an electronic medical record (EMR) associated with a patient and the actionable situation is a health situation.

The agent may be, for example, a system for handling EMRs. In such embodiments, the agent may be, for example, a software application executing on a computing device or a web-based application accessed by a user utilising a computing device coupled to a communications network.

In some embodiments, the actionable situation is a health situation based on at least one of a health condition, medical history, prescribed medication, and non-prescribed medication.

In some embodiments, the intervention performer is one of: a software application executing on a computing device, a web-based application, a chat bot, or a short message service (SMS) text message server. The intervention performed may be adapted to communicate with the agent, when the agent is a software application or web-based application.

In some embodiments, the agent data set is an EMR associated with a patient and the intervention performer is accessible by the patient, the intervention performer being adapted to display the intervention recommendation to the patient.

The method receives, at an intervention controller, an agent data set associated with an agent. Depending on the context, the agent may be, for example, a user, such as a patient. In the case in which the agent is a patient, the agent data set may be personal health information associated with that patient, such as an electronic medical record (EMR). Alternatively, the agent data set may be an EMR associated with a patient and the agent may be a system for handling and forwarding the EMR, such as an online computer program accessed by a health practitioner to create, modify, and upload EMRs. When the agent is a system for handling and forwarding the EMR, such a system may be medical practice software executing on a computing device.

The method and system utilise the health situation generator to analyse the received agent data set and determine if there is an actionable situation, based on the agent data set. For example, if a patient provides personal health information that indicates that the patient is taking multiple medications, the method may determine that an actionable situation exists, wherein the actionable situation relates to obtaining knowledge pertaining to each of the multiple medications.

In another example, for each medication that a patient is taking, the health situation generator determines whether there are any interaction alerts associated with that medication. If any interaction alerts are present, the health situation generator assesses the relevance of those alerts, such as based on the age or other conditions of the patient or other medications associated with the patient, and determines whether the interaction alerts correspond to an actionable situation for that patient.

The mere provision of an agent data set may be sufficient to indicate the presence of an actionable situation, wherein identification of the actionable situation includes parsing data in the agent data set to identify the actionable situation. For example, uploading an agent data set in the form of an EMR may be an indication, of itself, that a patient has presented to a health practitioner, such as a general practitioner or specialist. The EMR may include data stored in a predefined format, such that identifying an actionable situation, in the form of a health situation, is a matter of parsing the EMR to identify relevant content. Such relevant content may include, for example, a currently diagnosed condition, past medical history, prescribed medication, other medication, symptoms, age, and the like.

On determining the existence of an actionable situation, the method and system utilise the intervention controller to send notification of the actionable situation to at least one responder, wherein each responder has access to at least one source of information relating to the actionable situation. Each of the at least one responders may then choose whether or not to respond by returning an intervention recommendation to the intervention controller. The decision by a responder on whether or not to respond may be based, for example, on whether the respective responder has access to information that is pertinent to the actionable situation.

In one implementation, the system includes a set of responders corresponding to one or more databases that store information relating to medications. In another implementation, the intervention controller requests information pertaining to medications from responders in the form of one or more third-party online systems, such as databases maintained by pharmaceutical companies and regulatory authorities (e.g., the Therapeutic Goods Administration (TGA) of Australia or the Food and Drug Administration (FDA) of the United States of America). In a further implementation, the intervention controller polls a set of responders that include one or more of an internal database, an external database, or any combination thereof. The information pertaining to a medication may include any alerts, as mentioned above, along with information pertaining to drug to drug interactions, and cautions relating to age or medical conditions. A responder is not limited to a database, but may include other online data resources and may also include individuals. Depending on the implementation, such data resources may be accessed using simple queries or may include chat bots or the like implemented utilising artificial intelligence.

An intervention controller receives one or more intervention recommendations from one or more of the at least one responders and then determines whether to forward any one or more of the intervention recommendations to the agent. In the example in which the agent is a patient taking multiple medications, an intervention recommendation may be to stop one of the medications or substitute a medication, based on potential drug-drug interactions from the multiple medications. The determination of whether to forward the intervention recommendation may be based, for example, on one or more of the success of previous intervention recommendations provided by the responder, qualifications of the responder, recommendations from other responders, and matching of attributes associated with the responder to requirements specified in the agent data set. For example, the agent data set may include preferences or requirements specified by the agent or an end user that must be matched by a responder.

In one implementation, the intervention controller includes a programmed decision engine that makes the determination as to which, if any, intervention recommendation, is to be forwarded to the agent. As noted above, the programmed decision engine may utilise information pertaining to the responder, such as a stored record of past success rate, qualifications, and the like. The decision engine may also utilise a ranking of the respective responders. Depending on the implementation, the ranking may be user-defined, result from previous intervention recommendations, or a combination thereof. For example, where the method and system utilise a responder register, the method and system may rank the responders within the list, based on attributes of the respective responders. The decision engine optionally utilises that ranking when determining whether to forward an intervention recommendation from one of the responders.

In one example, the method and system are practised in relation to a healthcare environment. One embodiment provides for registering a user. One embodiment provides for receiving input indicative of Personal Health Information pertaining to the registered user. One embodiment provides for determining whether the received Personal Health Information indicates the presence of a Health Situation. In the case that the accepted Personal Health Information indicates the presence of a Health Situation, one embodiment provides for requesting an Intervention Recommendation from at least one registered Health Situation Responder. One embodiment provides for accepting from a Health Situation Responder an input indicative of an Intervention Recommendation. One embodiment provides for determining whether to perform an Intervention according to the accepted Intervention Recommendation.

Various embodiments are described throughout this specification with reference to the health care sector or medical industry. Such embodiments are illustrative and not restrictive and it will be readily understood that the method and system of the present disclosure may be equally practised in relation to other industrial sectors and industries.

### Terminology

Throughout this specification, the term “Personal Health Information” is an example of an agent data set that refers to medically relevant information that pertains to a particular individual. Some examples of Personal Health Information may include, without limitation, a prescription made out to that individual, biometric information about that individual, and the medical history of that individual, as well as medical prognoses and other medical predictions made for that individual by medical personnel and/or a computer-implemented agent, such as an artificial intelligence.

The term “Health Situation” is an example of an actionable situation that refers to a pre-determined non-empty set of Personal Health Information. That is, a Health Situation is a set of contextual information to be presented to a health responder. In on example, a Health Situation is an EMR associated with a patient. Depending on the circumstances, the EMR may include, for example, information about a health condition experienced by the patient, medical history of the patient, medicines being taken or prescribed to be taken by the patient, and the like.

The term “Intervention” refers to the presentation of medical knowledge to an individual, optionally including the acceptance of a response to the presented medical knowledge from that individual.

The term “Intervention Recommendation” refers to a specification according to which an Intervention can be performed.

The term “Health Situation Responder” is an example of a responder and refers to a computer-implemented agent capable of receiving input indicative of a Health Situation and responding to that input with an output indicative of an Intervention Recommendation.

### Overview

The method and system of the present disclosure utilise an Intervention Controller to acquire knowledge distributed among at least one source of information. The Intervention Controller is implemented using a computing device, such as a computer server, coupled to a communications network. One or more responders register with the Intervention Controller, wherein each responder has access to at least one source of information. In the example based in the healthcare industry, one or more Health Situation Responders register with the Intervention Controller, wherein each Health Situation Responder has access to at least one source of information. The at least one source of information may include, for example, but is not limited to, product data sheets, product information documents, databases, libraries, professional staff, and the like.

A user registers with the Intervention Controller and uploads, to the Intervention Controller, Personal Health Information pertaining to the registered user. A Health Situation Generator associated with the Intervention Controller parses the received Personal Health Information and determines whether the received Personal Health Information indicates the presence of a Health Situation. In one implementation, determining the presence of a Health Situation includes the application of a set of pre-determined rules to the Personal Health Information. The set of pre-determined rules may include, for example, at least one rule that determines the presence of a Health Situation when the statistical likelihood of an adverse health outcome for the user, in light of the Personal Health Information of that user, exceeds a pre-determined or predefined threshold. Other rules may relate, for example, to the presence of a prescribed medication, a diagnosed medical condition, or biometric parameter (e.g., age, blood pressure, haemoglobin level, etc.) with one or more predefined thresholds associated with each parameter.

When the Intervention Controller/Health Situation Generator determines the presence of a Health Situation, the Intervention Controller sends a request for an Intervention Recommendation to at least one registered Health Situation Responder. In one implementation, the Intervention Controller sends a request for an Intervention Recommendation to each registered Health Situation Responder. In another implementation, the Intervention Controller sends a request for an Intervention Recommendation to a selected subset of registered Health Situation Responders. Selection of the subset of registered Health Situation Responders may be based, for example, on a selection criteria, that may include, but is not limited to, one or more of past responses, past outcomes, personal preferences associated with the patient, or any combination thereof. In another implementation, the Intervention controller sends a request to one registered Health Situation Responder which, based on rules, also forwards the request to other Health Situation Responders for consideration.

The Intervention Controller receives input indicative of an Intervention Recommendation from a registered Health Responder and then determines whether or not to perform an intervention based on the received Intervention Recommendation or whether to send additional requests to additional responders. When the Intervention Controller determines that an intervention is to be performed, the Intervention Controller performs an intervention in accordance with the Intervention Recommendation.

In one embodiment, registering a Health Situation Responder with the Intervention Controller includes the following steps:
a. Receiving a Registration Request from the Health Situation Responder;
b. Determining whether to allow or deny the Registration Request;
c. In case it is determined that the Registration Request is to be allowed, storing Registration Information for the Health Situation Responder in a storage subsystem associated with the Intervention Controller; and
d. Signalling to the Health Situation Responder whether the Registration Request has been accepted or denied.

In one embodiment, registering a user with the Intervention Controller includes the following steps:
a. Accepting input of User Information; and
b. Storing the User Information in the storage subsystem.

In one embodiment, the method and system generate at least one Reference Persona for a user registering with the Intervention Controller and store the generated Reference Persona(s) in a storage subsystem associated with the Intervention Controller. Depending on the implementation, a patient may be associated with multiple Reference Personas as a result of visiting different medical practices, wherein a different Reference Persona is generated in relation to each respective medical practice. Different Reference Personas may also be generated to accommodate different levels of anonymity that are required or preferred to be utilised by different responders.

In one embodiment, generating a Reference Persona includes:
a. Selecting at least one part of the User Information, the selected part being sufficient to uniquely identify the User;
b. Optionally converting the part of the User Information to a canonical format;
c. Using a one-way cryptographic hash function to generate for each selected part of the User Information a corresponding Reference Value; and
d. Using the Reference Values to populate the corresponding attributes values of the Reference Persona.

Personal Health Information provided by the registered user to the Intervention Controller may include any personal health information associated with that user and may include, for example, but is not limited to, one or more of: a prescription made out to the user; medication the user is currently taking; medication the user has taken in the past; the user's consumption of medically relevant substances; biometric information pertaining to the user; and information pertaining to medically relevant behaviours on the part of the user.

In one embodiment, requesting an Intervention Recommendation includes sending an Intervention Recommendation Request from the Intervention Controller to at least one registered Health Situation Responder. In one implementation, the Intervention Recommendation Request includes a Reference Persona associated with the user and Health Situation Information associated with the user.

In one embodiment, determining whether to execute an Intervention according to an accepted Intervention Recommendation includes at least one of:
a. Assessing the trustworthiness of the Intervention Recommendation;
b. Assessing the likelihood that the user will engage with the Recommendation;
c. Assessing the benefit to the User of running the Intervention;
d. Assessing the severity of the Health Situation; and
e. Assessing the financial incentive for running the Intervention.

In one embodiment, executing an Intervention includes the Intervention Controller sending the Intervention Result to the Health Situation Responder that generated the Intervention Recommendation according to which the Intervention was executed. In one implementation, executing the Intervention further includes processing financial incentives for executing the Intervention;

In one embodiment, registering a Health Situation Responder includes processing financial incentives for registering the Health Situation Responder.

In one embodiment, the Intervention Controller accepts input indicative of an Adverse Health Outcome. The Adverse Health Outcome may be associated with an earlier Intervention Recommendation received from a Health Responder. The input may be received from a user providing input to the system, such as by utilising a user interface of an app or website associated with the system, or by sending a Short Message Service (SMS) text message to a predefined number associated with the Intervention Controller. Alternatively, the input may be received within an EMR uploaded to the system, such as may be generated in association with a follow up visit to a medical practice. For example, following an Intervention Recommendation, a patient may present to a general practitioner (GP) with a side effect, such as a rash. The GP updates the EMR to note the rash and an EMR is forwarded to the Intervention Controller.

Following input indicative of an Adverse Health Outcome, the Intervention Controller may optionally send an Adverse Health Outcome Report to registered Health Situation Responders. Depending on the implementation, the Intervention Controller sends the Adverse Health Outcome Report to the Health Situation Responder that had previously generated an Intervention Recommendation. Alternatively, the Intervention Controller sends the Adverse Health Outcome Report to all Health Situation Responders that responded to the Health Situation or to a selected subset of all Health Situation Responders. For example, the Intervention Controller optionally sends an Adverse Health Outcome Report to all Health Situation Responders that have particular capabilities, qualifications, or store data relating to a prescribed medication that was associated with the Adverse Health Outcome. In a further alternative, the Intervention Controller sends the Adverse Health Outcome Report to all registered Health Situation Responder.

In one embodiment, the method and system cache an accepted Intervention Recommendation.

Different embodiments each include a computer-readable and tangible carrier medium on which are encoded instructions that when executed by one or more processors of a processing system are operable to carry out any of the method embodiments described herein. Particular embodiments may provide all, some, or none of these aspects, features, or advantages. Particular embodiments may provide one or more other aspects, features, or advantages, one or more of which may be readily apparent to a person skilled in the art from the descriptions, drawings, and claims herein.

### Principal Components

The method and system of the present disclosure utilise a central server that implements an Intervention Controller to facilitate the acquisition of knowledge from at least one source. Users registered with the Intervention Controller are able to submit requests for information. On receipt of such a request from a registered user, the Intervention Controller notifies a set of registered Responders and each Responder then has the option of responding to the request.

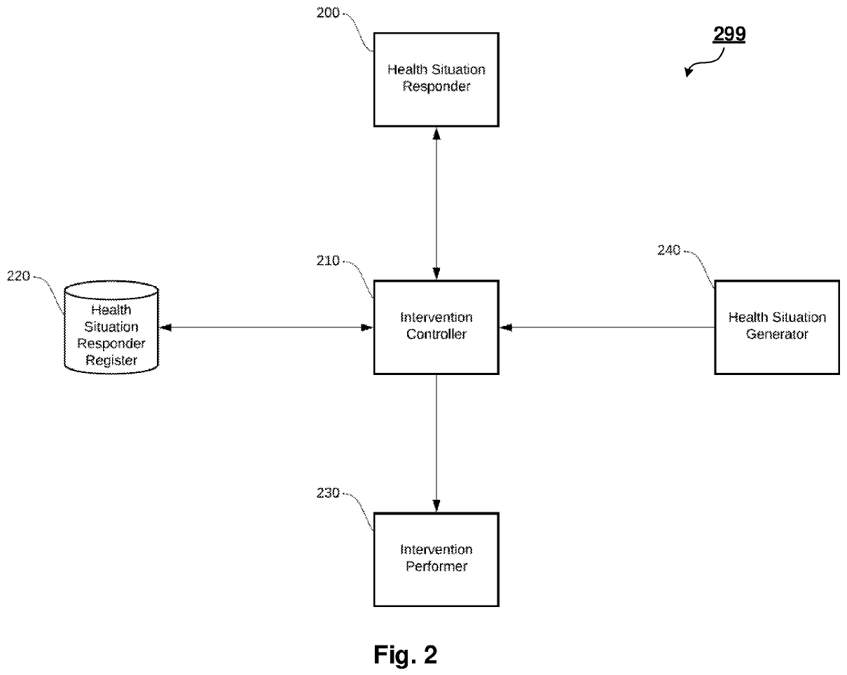

FIG. 2 is a schematic block diagram representation of a system 299 for acquiring medical knowledge from at least one source. In the example of FIG. 2, the system 299 includes an Intervention Controller 210 that maintains a Health Situation Responder Register 220. The Intervention Controller 210 may be implemented, for example, using a computer server coupled to a communications network. The communications network may comprise one or more wired communications links, wireless communications links, or any combination thereof. In particular, the communications network 205 may include a local area network (LAN), a wide area network (WAN), a telecommunications network, or any combination thereof. A telecommunications network may include, but is not limited to, a telephony network, such as a Public Switch Telephony Network (PSTN) or a cellular mobile telephony network, the Internet, or any combination thereof.

The Health Situation Responder Register 220 is a stored list of available Health Situation Responders, wherein each Health Situation Responder is an entity that has access to at least one source of electronic medical information. In one implementation, the Health Situation Responder Register 220 is stored in a computer readable storage medium associated with the Intervention Controller 210 and may be integrated with, co-located with, or remotely coupled to a computing device implementing the Intervention Controller 210.

In one implementation, only Health Situation Responders registered with the Intervention Controller 210 appear on the Health Situation Responder Register 220. In an alternative implementation, an administrator of the Intervention Controller 210 enters one or more Health Situation Responders to the Health Situation Responder Register 220. For example, the Intervention Controller 210 may send a request to a public facility or database or to a known medical institute, university, or the like, and enters the relevant facility to the Health Situation Responder Register 220.

In one embodiment, the Health Situation Responder Register 220 is a set of entries, each entry representing one Health Situation Responder and forming a Health Situation Responder Profile associated with a corresponding Health Situation Responder. Each Health Situation Responder Profile stores information pertaining to the corresponding Health Situation Responder and may include, for example, but is not limited to, one or more of:
a. Information required to authenticate a Registration Request from the represented Health Situation Responder;
b. Information indicative of whether the Intervention Controller 210 is to accept a future Registration Request from the represented Health Situation Responder;
c. Information indicative of whether the Intervention Controller 210 has received a Registration Request from the represented Health Situation Responder and whether that Registration Request was accepted by the Intervention Controller 210; and
d. In case the represented Health Situation Responder has made a

Registration Request that has been accepted by the Intervention Controller 210, information indicative of an instruction to the Intervention Controller 210 for how the Intervention Controller 210 is to report Health Situations to said represented Health Situation Responder.

The Health Situation Responder Register 220 may be implemented in many ways, depending on the particular application. In one implementation, the Health Situation Responder Register 220 is a text-file stored in JavaScript Object Notation (JSON) format. In another implementation, the Health Situation Responder Register 220 is a table in a relational database. It will be appreciated that many alternative implementations may be utilised for the Health Situation Responder Register 220 without departing from the spirit and scope of the present disclosure.

In the example of FIG. 2, the Health Situation Responder Register 220 includes a single Health Situation Responder 220 and the system 299 further includes a Health Situation Generator 240 and an Intervention Performer 230.

According to one implementation, the instruction for how the Intervention Controller 210 is to report a Health Situation to a Health Situation Responder is implemented using a text string representing the URL of a web API end-point to which a call can be made using Representational State Transfer (REST). Alternatively, Remote Procedure Calls (RPCs) could be utilised to effect communication. Similar communication methods, or combinations thereof, may be utilised for other communication exchanges, such as between the agent and the Intervention Controller. Those skilled in the art will appreciate that many alternative implementations may equally be practised.

When the Intervention Controller 210 receives Personal Health Information from a patient, the Intervention Controller 210 invokes the Health Situation Generator 240, which parses the Personal Health Information in order to determine if there is a Health Situation. If there is a Health Situation, the Intervention Controller 210 sends reports of the Health Situation to one or more Health Situation Responders 200 in the Health Situation Responder Register 220.

The example of FIG. 2 shows a single Health Situation Responder 200. The Health Situation Responder 200 accepts reports of Health Situations from the Intervention Controller 210. On receiving a report of a Health Situation, the Health Situation Responder determines a Health Situation Response, being one of:
a. Returning an Intervention Recommendation to the Intervention Controller 210, in case there is at least one Intervention that the Health Situation Responder 200 deems appropriate in relation to the reported Health Situation; and
b. Returning no Intervention Recommendation to the Intervention Controller 210, in case there is no Intervention the Health Situation Responder 200 deems appropriate in relation to the reported Health Situation.

Determining the Health Situation Response may be performed in many different ways, according to the particular implementation and use case. In one implementation, determining the Health Situation Response is done according to a set of predefined rules. In an alternative implementation, determining the Health Situation is done by operating an artificial intelligence that has been trained to recognise appropriate interventions for a given set of Health Situations. In a further alternative implementation, the Health Situation Response is done by a suitably trained human operator who interacts with the Health Situation Responder 200 via a suitable user interface of a computing device.

In one embodiment, the determined Intervention Recommendation further includes a confidence assessment, indicative of how confident the Health Situation Responder 200 is that the determined Intervention Recommendation is correct for the reported Health Situation. One exemplary confidence assessment is a numerical value between 0 and 1, where 0 means no confidence, and 1 means certainty that the Intervention Recommendation is correct. An alternative confidence assessment is a colour associated with a returned Intervention Recommendation, wherein different colours represent a different confidence level. For example, green may indicate a high level of confidence that the Intervention Recommendation is correct for the reported Health Situation, with red indicating a low level of confidence and yellow indicating an average level of confidence. In one implementation, a confidence assessment returned with an Intervention Recommendation includes a combination of a colour and a numerical value.

In one embodiment, the determined Intervention Recommendation further includes an importance assessment, indicative of how important the Health Situation Responder 200 considers the performance of the Intervention specified in said Intervention Recommendation to be. One exemplary importance assessment is the value “Mandatory” meaning the intervention must be performed. For example, an Intervention Recommendation relating to an identified drug-drug interaction (DDI) may be identified as a mandatory intervention. Another exemplary importance assessment is “Optional” meaning the Intervention Recommendation may be safely ignored. For example, a pricing comparison relating to medications prescribed for a user may be identified as an optional intervention. A further exemplary importance assessment is “Recommended”, indicating that the intervention is recommended, but not essential.

According to one embodiment, on receiving a Health Situation Response from the Health Situation Responder 200, the Intervention Controller 210 determines whether the Intervention Recommendation in said Health Situation Response is to be accepted. In one embodiment, the default action is to accept automatically an Intervention Recommendation received from a Health Situation Responder 200. In one embodiment, the process of making this determination includes at least one of:
a. Accepting the Intervention Recommendation only if the confidence assessment provided by the Health Situation Responder 200 exceeds a pre-determined minimum confidence threshold value; and
b. In case there are multiple Health Situation Responses from multiple Health Situation responders, accepting only those Intervention Recommendations for which the importance assessment exceeds a certain importance threshold value.

In one embodiment, the Intervention Controller 210 records Health Outcomes associated with past Intervention Recommendations and uses the recorded Health Outcomes to determine whether to accept a newly received Intervention Recommendation. Such Health Outcomes may include, for example, but are not limited to, readmission, further prescriptions, change of dosage, or a period of time elapsing without further intervention.

In case the Intervention Controller 210 accepts an Intervention Recommendation, the Intervention Controller 210 forwards the Intervention Recommendation to an Intervention Performer 230.

In one embodiment, the Intervention Performer 230 is one of a mobile app, a web-based app, a chat bot, SMS text message server, or the like, by which the Intervention Controller 210 is able to communicate a selected Intervention Recommendation to the agent. In the scenario in which there are multiple Intervention Performers 230, the Intervention Controller 210 selects an Intervention Performer 230. Selection of an Intervention Performer 230 may be made in accordance with performance criteria or, alternatively, made based on availability to the agent. The Intervention Performer 230 may be, for example, a means by which to inform the agent of the Intervention Recommendation, in which case the Intervention Performer 230 may be one or more of an app, web application, chat bot, SMS text message, or the like.

In one embodiment, the Intervention Performer 230 is an automated agent that interacts with users by sending text messages, for example SMS text messages, emails, or in-app messages.

### Performing an Intervention

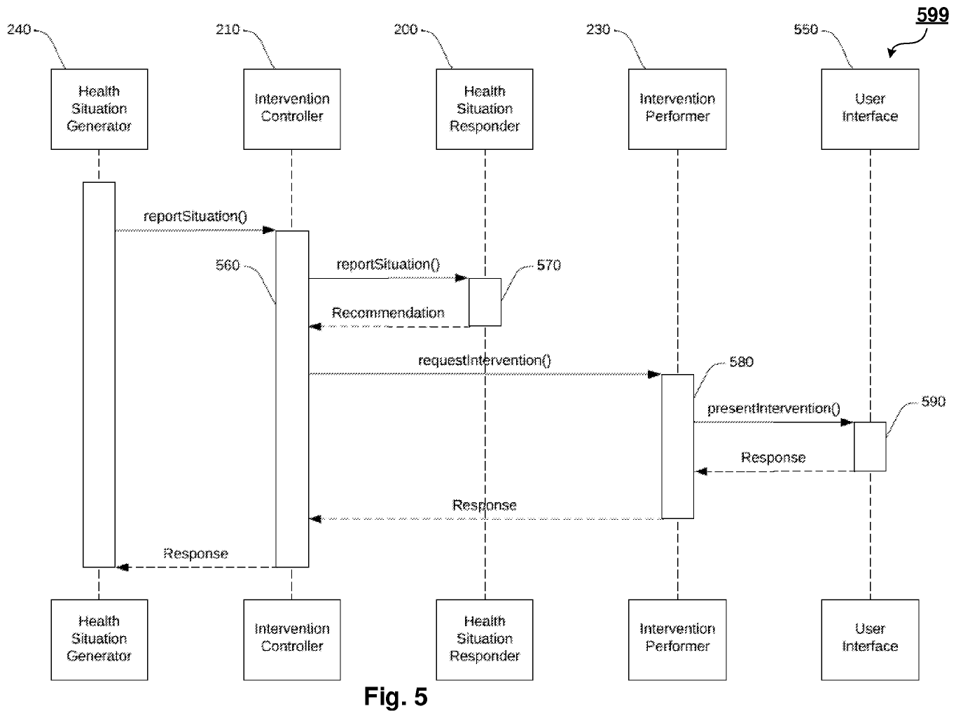

FIG. 5 depicts, in simplified form, a sequence diagram 599 illustrating some of the method invocations involved in the generation and processing of a Health Situation. Those skilled in the art will appreciate that method and entity names are by way of example only. Other embodiments with different names may equally be practised.

In the example of FIG. 5, the Health Situation Generator 240 has determined that the conditions for a particular Health Situation have been satisfied, based on a received agent data set in the form of Personal Health Information pertaining to a patient. The Health Situation Generator 240 informs the Intervention Controller 210 of having identified a Health Situation by invoking a reportSituation method 560 of the Intervention Controller 210 and includes the Health Situation as an invocation parameter.

The Intervention Controller 210 now determines if there is at least one registered Health Situation Responder 200 to which the Health Situation is to be reported, and in case there is at least one such Health Situation Responder 200 invokes the reportSituation method 570 of each such Health Situation Responder 200 and includes the Health Situation as an invocation parameter.

According to one embodiment, Intervention Controller 210 simply invokes the reportSituation invocation 570 for each registered Health Situation Responder 200. According to an alternative embodiment, the step of determining to which Health Situation Responder 200 the Health Situation is to be reported includes the evaluation of a set of pre-determined business rules. One exemplary business rule is that the Health Situation Responder has been given the relevant security clearance for the particular Health Situation and has declared its interest in receiving reports about the Health Situation as part of its registration with the Intervention Controller 210.

The Health Situation Responder 200 now evaluates the Health Situation and returns to the Intervention Controller 210 an Intervention Recommendation, that Intervention Recommendation being depicted in FIG. 5 using the term “Recommendation”.

For some Health Situations, the Intervention Recommendation may be not to perform any Intervention. For example, the Health Situation may have been identified on the basis of the Personal Health Information including multiple medications, but the Health Responder may have determined, based on at least one source of information, that there are no adverse drug to drug interactions for those multiple medications. For other Health Situations, the Intervention Recommendation may be to perform an intervention.

In one embodiment, the Health Situation Responder 200 has a set of pre-determined rules that determine which Intervention to recommend, if any, for each Health Situation to which the Health Situation Responder 200 is responsive.

Those skilled in the art will appreciate that other embodiments of the present invention exist wherein the Health Situation Responder 200 makes this determination according to other means, for example, by using an artificial intelligence to determine the Intervention Recommendation. In one embodiment, a combination of such means is used.

In case the Intervention Recommendation is that no Intervention is to be performed, then the Intervention Controller 210 returns a Response to the Health Situation Generator 240 indicating that no Intervention was performed.

In some embodiments, the Intervention Controller 210 now determines whether to accept or ignore the recommendation. This determination may be based, for example, on a confidence level associated with, or included in, the Intervention Recommendation, as provided by the Health Situation Responder 200.

In case the Intervention Controller 210 determines to ignore the Intervention Recommendation, the Intervention Controller 210 returns a Response to the Health Situation Generator 240 indicating the Intervention Recommendation, as well as the decision to ignore the Intervention Recommendation.

In case the Intervention Controller 210 determines that the Intervention is to be performed, the Intervention Controller invokes the requestlntervention method 580 of the Intervention Performer 230.

The Intervention Performer 230 invokes the presentlntervention method 590 on a User Interface 550 on a computing device accessed by the user, wherein the User Interface 550 displays the Intervention to be performed to the user, and optionally accepts a response from the user. Such a response may be received, for example, using a dialog box text field, or selection of one or more predefined responses selectable by check-boxes, radio buttons, drop-down lists, or other input devices.

According to one embodiment, the Intervention Performer is a mobile app executing on a computing device accessed by the user and the User Interface 550 is the graphical user interface of the app. According to another embodiment, the Intervention Performer is a computer-implemented process that sends a text message (e.g., an SMS) or in-app message to a device operated by the user, e.g., a mobile phone. Those skilled in the art will appreciate that many embodiments exist for performing interventions.

In case the User Interface 550 accepts a Response from the user and the user provides a Response, then the User Interface 550 provides a signal to the Intervention Performer 230 indicative of the Response provided by the user.

In case no Response is accepted by the User Interface 550 or the user has declined to provide a Response, once the intervention has been completed, the User Interface 550 returns a signal to the Intervention Performer 230 indicating that the Intervention has been completed.

The Intervention Performer 230 returns a signal to the Intervention Controller 210 indicative of the signal returned by the User Interface 550. The Intervention Controller 210 then returns a signal to the Health Situation Generator 240 indicative of the signal return by the Intervention Performer 230.

According to some embodiments of the present disclosure, the Intervention Controller 210 performs an additional step of generating an Outcome Classification. That is, the Intervention Controller 210 classifies the user's Response to a performed Intervention. In one exemplary embodiment, the user's Response is classified as being either positive or negative. In alternative embodiment, the user's Response is classified according to a numerical scale, for example from 1 to 10, where 1 is a least desirable outcome and 10 is a most desirable outcome.

Having classified the user's Response, the Intervention Controller 210 stores information about the performed Intervention, along with the Outcome Classification. Storing such information can be used to improve the quality of decisions made by the Intervention Controller 210, for example when deciding whether to perform or ignore an Intervention Recommendation, such decisions being made according to pre-determined business rules in some exemplary embodiments and according to the determination of artificial intelligences trained on that information in other exemplary embodiments.

In one embodiment, the Intervention Controller 210 returns a signal indicating the Outcome Classification for each Intervention that was performed according to an Intervention Recommendation to the Health Situation Responder 200 that generated the Intervention Recommendation.

In one embodiment, the Intervention Controller includes the Outcome Classification to the Health Situation Generator 240 with the Response signal.

### Presenting an Intervention

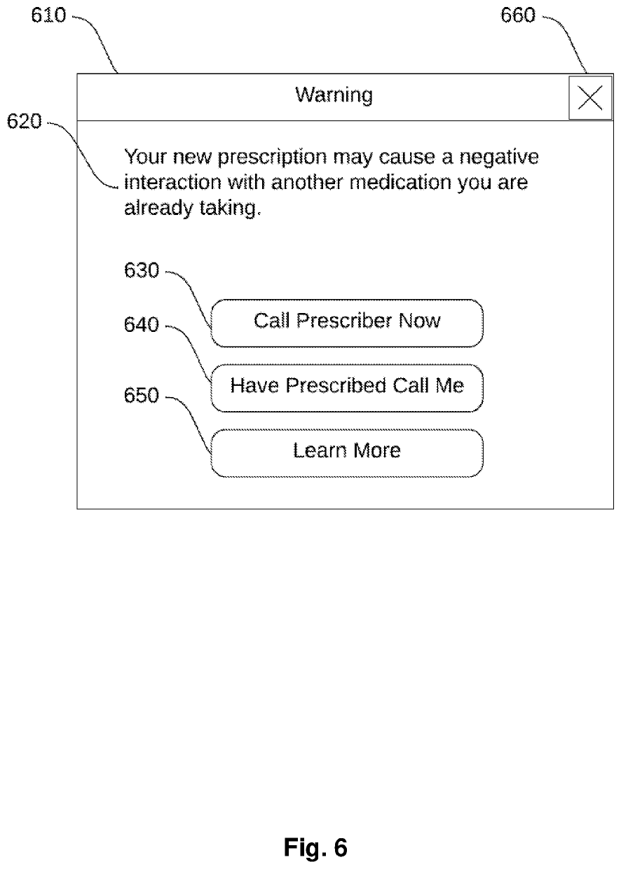

FIG. 6 illustrates, in simplified form, an exemplary dialog box 610 presented by the User Interface 550 when performing an Intervention. In this example, the dialog box 610 displays a Warning Message 620 to the user:
“Your new prescription may cause a negative interaction with another medication you are already taking.”

According to this example, the User Interface 550 accepts four possible actions from the user. The first exemplary action is to press button 630 to invoke a dialler and call the prescriber. The second exemplary action is to press button 640 to invoke a process for scheduling a callback from the prescriber. The third exemplary action is to press button 650 to invoke the presentation of additional information (e.g., text, audio, and/or video) in relation to Warning Message 610. The final exemplary action is to press the Dialog Close Button 660 and close the dialog box 610, in order to decline a Response to the Intervention.

Computer Implementation

Unless specifically stated otherwise, as apparent from this description, it is appreciated that throughout the specification discussions utilising terms such as “processing,” “computing,” “calculating,” “determining”, or the like, may refer to, without limitation, the action and/or processes of hardware (e.g., an electronic circuit, a computer or computing system, or similar electronic computing device) that manipulate and/or transform data represented as physical, such as electronic, quantities into other data similarly represented as physical quantities.

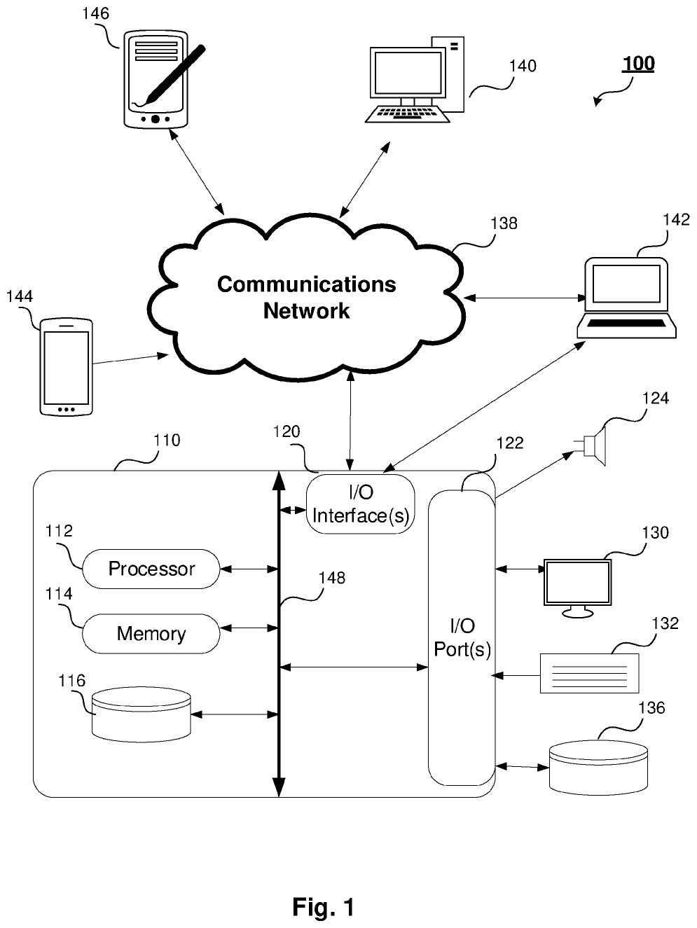

The system of the present disclosure for passively acquiring knowledge distributed among at least one source of information may be practised using one or more computing devices, such as a general purpose computer or computer server, programmed to provide customised and improved computing devices configured to perform the methods described herein. FIG. 1 is a schematic block diagram representation of a system 100 that includes a general purpose computer 110. The general purpose computer 110 includes a plurality of components, including: a processor 112, a memory 114, a storage medium 116, input/output (I/O) interfaces 120, and input/output (I/O) ports 122. Components of the general purpose computer 110 generally communicate with each other using one or more buses 148.

The processor 112 be implemented using any device or portion of a device that processes electronic data (e.g., from registers and/or memory to transform that electronic data into other electronic data that may be stored in registers and/or memory). The processor 112 may be representative of one or more processors operating individually, sequentially, or in parallel

The memory 114 may be implemented using Random Access Memory (RAM), Read Only Memory (ROM), or a combination thereof. The storage medium 116 may be implemented as one or more of a hard disk drive, a solid state “flash” drive, an optical disk drive, or other storage means. The storage medium 116 may be utilised to store one or more computer programs, including an operating system, software applications, and data. In one mode of operation, instructions from one or more computer programs stored in the storage medium 116 are loaded into the memory 114 via the bus 148. Instructions loaded into the memory 114 are then made available via the bus 148 or other means for execution by the processor 112 to implement a mode of operation in accordance with the executed instructions.

One or more peripheral devices may be coupled to the general purpose computer 110 via the I/O ports 122. In the example of FIG. 1, the general purpose computer 110 is coupled to each of a speaker 124, a display device 130, an input device 132, and an external storage medium 136. The speaker 124 may be implemented using one or more speakers, internal to the computing device 110 or external to the computing device 110, such as in a stereo or surround sound system. In the example in which the general purpose computer 110 is utilised to implement a user computing device having a user interface 550 in accordance with FIG. 5, one or more peripheral devices may relate to a display, keyboard, mouse, stylus, or other input or output device connected to the I/O ports 122.

The display device 130 may be a computer monitor, such as a cathode ray tube screen, plasma screen, or liquid crystal display (LCD) screen. The display 130 may receive information from the computer 110 in a conventional manner, wherein the information is presented on the display device 130 for viewing by a user. The display device 130 may optionally be implemented using a touch screen to enable a user to provide input to the general purpose computer 110. The touch screen may be, for example, a capacitive touch screen, a resistive touchscreen, a surface acoustic wave touchscreen, or the like.

The input device 132 may be a keyboard, a mouse, a stylus, drawing tablet, or any combination thereof, for receiving input from a user. The external storage medium 136 may include an external hard disk drive (HDD), an optical drive, a floppy disk drive, a flash drive, solid state drive (SSD), or any combination thereof and may be implemented as a single instance or multiple instances of any one or more of those devices. For example, the external storage medium 136 may be implemented as an array of hard disk drives.

The I/O interfaces 120 facilitate the exchange of information between the general purpose computing device 110 and other computing devices. The I/O interfaces may be implemented using an internal or external modem, an Ethernet connection, or the like, to enable coupling to a transmission medium. In the example of FIG. 1, the I/O interfaces 122 are coupled to a communications network 138 and directly to a computing device 142. The computing device 142 is shown as a personal computer, but may be equally be practised using a smartphone, laptop, or a tablet device. Direct communication between the general purpose computer 110 and the computing device 142 may be implemented using a wireless or wired transmission link.

The communications network 138 may be implemented using one or more wired or wireless transmission links and may include, for example, a dedicated communications link, a local area network (LAN), a wide area network (WAN), the Internet, a telecommunications network, or any combination thereof. A telecommunications network may include, but is not limited to, a telephony network, such as a Public Switch Telephony Network (PSTN), a mobile telephone cellular network, a short message service (SMS) network, or any combination thereof. The general purpose computer 110 is able to communicate via the communications network 138 to other computing devices connected to the communications network 138, such as the mobile telephone handset 144, the touchscreen smartphone 146, the personal computer 140, and the computing device 142.

One or more instances of the general purpose computer 110 may be utilised to implement a server acting as a data acquisition system in accordance with the present disclosure. In such an embodiment, the memory 114 and storage 116 are utilised to store data relating to agent records, registered responders, adverse outcomes, historical data, user data, and user interface templates. Software for implementing the data acquisition system is stored in one or both of the memory 114 and storage 116 for execution on the processor 112. The software includes computer program code for implementing method steps in accordance with the method for passively acquiring data described herein.

Embodiments of the present disclosure are not limited to any particular implementation or programming technique and the invention may be implemented using any appropriate techniques for implementing the functionality described herein. Furthermore, embodiments are not limited to any particular programming language or operating system.

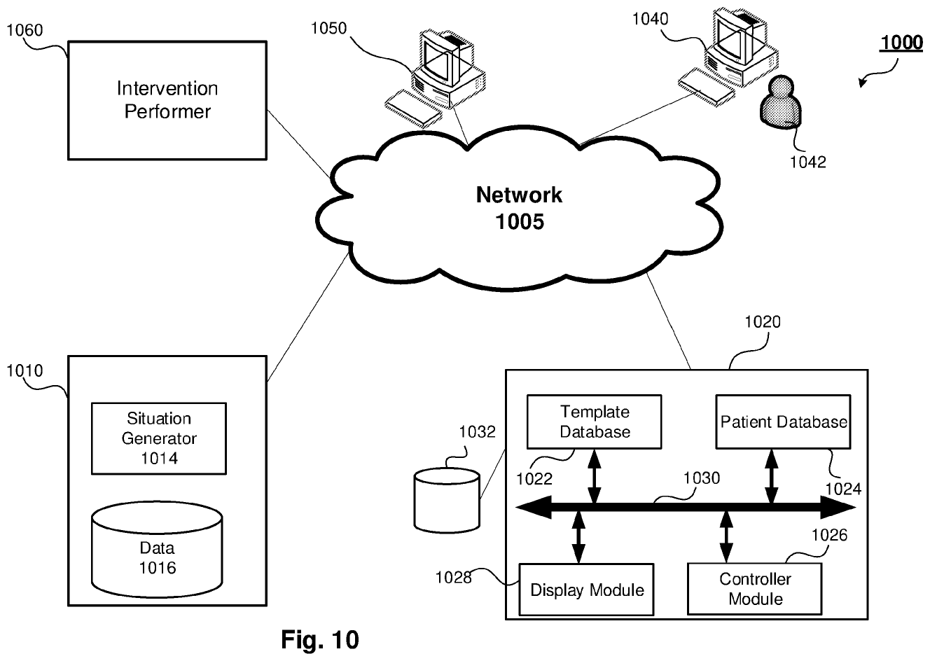

FIG. 10 is a schematic block diagram representation of a system 1000 for passively acquiring knowledge from at least one source of information, on which one or more embodiments of the present disclosure may be practised. In the example of FIG. 10, the system is a medical healthcare system that includes an intervention controller 1020 implemented using a custom-programmed computer server 1020 coupled to a communications network 1005.

The communications network 1005 may comprise one or more wired communications links, wireless communications links, or any combination thereof. In particular, the communications network 1005 may include a local area network (LAN), a wide area network (WAN), a telecommunications network, or any combination thereof. A telecommunications network may include, but is not limited to, a telephony network, such as a Public Switch Telephony Network (PSTN) or a cellular mobile telephony network, the Internet, or any combination thereof.

The intervention controller 1020 includes a template database 1022 for storing templates associated with a user interface to be presented to a user accessing the intervention controller 1020 via a web-based browser. The intervention controller 1020 also includes a patient database 1024 for storing information pertaining to registered patients, a display module for organising and transmitting information to be displayed by an entity accessing the intervention controller 1020, and a controller module 1026 for handling incoming patient information, health situations, and intervention recommendations. The intervention controller 1020 also includes a database 1032, which may be internal or external to the intervention controller 1020, and which stores data relating to past interventions and the like.

The database 1032 optionally stores information pertaining to medications and treatments, including any associated side effects, contraindications, drug to drug interactions, and the like. Contraindications may include, for example, but are not limited to, indications that caution should be used when a pharmaceutical or treatment is used in combination with one or more other pharmaceuticals or treatments, or indications that use of a pharmaceutical or treatment could have severe consequences, including death, if prescribed to someone with allergies, high blood pressure, pregnancy, or other condition. The database 1032 optionally stores information pertaining to previous intervention recommendations and outcomes relating to previous intervention recommendations, including subsequent treatments, symptoms, and medications.

The system 1000 also includes an agent computing device 1040 accessed by a medical practitioner 1042 and coupled to the communications network 1005. The medical practitioner 1042 may be, for example, a doctor, nurse, administrative assistant, or other personnel associated with a medical practice. The agent computing device 1040 may be a general purpose computer, such as the computer 110 of FIG. 1, or a smartphone, tablet, phablet, laptop computer, or any other suitable computing device. In this example, the agent computing device 1040 is running a software application for managing EMRs. The medical practitioner 1042 uses the computing device 1040 to communicate with the intervention controller 1020 via the network 1005 and register with the intervention controller 1020. In this way, the medical practitioner 1042 is able to upload agent data sets in the form of EMRs from the agent computing device 1040 to the intervention controller 1020. Details derived from the uploaded EMRs are stored in the patient database 1024.

The medical practitioner 1042 uploads an EMR containing patient information to the intervention controller 1020, which receives the EMR and then forwards the received EMR to a health situation generator 1010 via the communications network 1005. In the example of FIG. 10, the health situation generator 1010 is a separate entity from the intervention controller 1020. In an alternative implementation, the health situation generator 1010 is integral to, or co-located with, the intervention controller 1010. In one implementation, the intervention controller 1020 and situation generator 1010 are implemented using a cloud-based platform, such as Amazon Web Services (AWS), Microsoft Azure, Google Cloud, IBM Cloud, Oracle Cloud, or the like.

In the example of FIG. 10, the health situation generator 1010 includes a situation generator module 1014 and a generator database 1016. The health situation generator 1010 may be implemented using a general purpose computer 110 executing customised computer program instructions based on a set of predefined parsing rules. The situation generator module 1014 processes the EMR received from the intervention controller 1020 and identifies a health situation from patient information contained in the received EMR. In one embodiment, the situation generator module 1014 executes code that parses the EMR to identify a current health situation, such as a medical condition, prescribed medication or other treatment, past interventions, and the like. Depending on the implementation, the situation generator module 1014 identifies a health situation based on a data formatting and retrieval system, retrieving contextual information that is assembled and presented in the EMR in a standardised format.

As described in relation to the database 1032, the generator database 1016 optionally stores information pertaining to medications and treatments, including any associated side effects, contraindications, drug to drug interactions, and the like. Contraindications may include, for example, but are not limited to, indications that caution should be used when a pharmaceutical or treatment is used in combination with one or more other pharmaceuticals or treatments, or indications that use of a pharmaceutical or treatment could have severe consequences, including death, if prescribed to someone with allergies, high blood pressure, pregnancy, or other condition. The situation generator 1014 optionally references the generator database 1016 in order to identify a current health situation.

The health situation generator 1010, on determining the existence of a health situation, notifies the intervention controller 1020 of the health situation. The controller module 1026 then notifies a set of one or more health situation responders of the health situation, wherein each health situation responder has access to at least one source of information. In the example of FIG. 10, the system 1000 includes a single health situation responder 1050, implemented using a computing device 1050 coupled to the communications network 1005. The health situation 1050 responder is optionally registered in a health situation responder register associated with the intervention controller 1020. Alternatively, the intervention controller 1020 sends notifications of the health situation to health responders that are derived from an external database or directory listing.

The health situation responder 1050 receives the health situation notification and determines whether or not to respond to the received health situation notification. If the health situation responder 1050 determines, based on at least one information source to which it has access, that an intervention is required, the health situation responder 1050 sends an intervention recommendation to the intervention controller 1020. Such an intervention may be required, for example, when the health situation responder 1050 determines from the received health situation notification that there is a potential drug to drug interaction between two medications prescribed for the patient 1042. The information source(s) to which the health responder 1050 has access may include one or more data stores, such as electronic databases of diseases, medical conditions, anatomy, pharmaceutical product information, and the like. The information source(s) may also include access to one or more practitioners, such as professionals or other qualified persons able to respond to the actionable health situation. In a further alternative, the information source(s) may include a combination of data stores and human practitioners.

The health responder 1050 may be implemented using a computing device programmed to access an associated storage device on receipt of a health situation. Such a computing device optionally utilises artificial intelligence to determine whether an intervention is required, based on a received health situation. When an intervention is required, the artificial intelligence may be utilised to determine an intervention recommendation. The artificial intelligence may include, for example, a neural network trained on a data set of known questions and answers relating to one or more of medical products, diagnoses, treatments, and the like. An alternative implementation utilises human input, alone or in combination with artificial intelligence or other computer-related search tools to search one or more data sources associated with the health responder 1050 to determine whether an intervention is required and, if so, what intervention recommendation is to be made.

The health responder 1050 optionally sends a confidence assessment associated with any intervention recommendation forwarded to the intervention controller 1020. The confidence assessment may be based, for example, on a distance measure between a query projected into a data source associated with the health responder 1050 and populated nodes of that data source, indicating how close the query is to a known answer. Further, the health responder 1050 optionally sends an importance assessment associated with any intervention recommendation, wherein the importance assessment indicates how important it is that the intervention recommendation be followed. As noted above, importance assessments may include, for example, mandatory, optional, and/or recommended.

The controller module 1026 determines whether or not to act on the received intervention recommendation. This determination may be based, for example, on a confidence level associated with, or included with, the intervention recommendation. The determination may also be determined based on qualifications or other attributes associated with the responder that provided the intervention recommendation, including the success or otherwise of past intervention recommendations received from that responder. When the controller module 1026 determines that intervention is required, the controller module 1026 sends the intervention recommendation to an intervention performer 1060. The intervention performer 1060 is then tasked with performing the recommended intervention. In one example, the intervention performer 1060 interacts with an app executing on the computing device 1040 to message the medical practitioner 1042 to advise a patient associated with the EMR to stop taking one or more medications, to change a medication, or to see a doctor. Other interventions may include, for example, but are not limited to, automated follow-up communications, a link back to patient care, a switch to generic or alternative medications with fewer side effects, changing dosages or treatment regime, and the like.

In an alternative implementation, the intervention performer 1060 is adapted to communicate with a user computing device (not shown) accessed by the patient associated with the EMR. In this implementation, the intervention performer 1060 may be a software application that sends information to the user computing device, wherein the software application may present a user interface to the display of the user computing device or otherwise act as a chat bot, messaging device, or the like. Alternatively, the intervention performer 1060 may be an SMS messaging server adapted to send text messages to a phone associated with the patient. In a further alternative, the intervention performed 1060 sends a link to the user computing device, wherein the link connects the user computing device to a webpage or the like through which the intervention performed 1060 is able to communicate with the user computing device accessed by the patient.

### Registering a Health Situation Responder

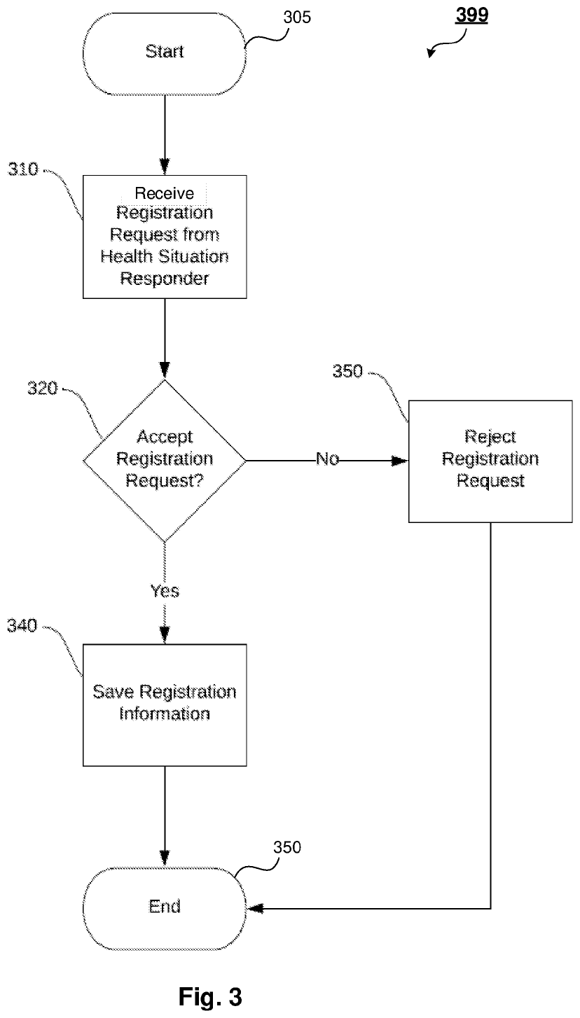

FIG. 3 is a flow diagram that illustrates, in simplified form, a method 399 for registering a Health Situation Provider with an Intervention Controller 210. The method 399 begins at a Start step 305 and proceeds to step 310, in which the Intervention Controller receives a registration request from a Health Situation Responder 200.

In one embodiment, the registration request includes at least some of the following:
a. Authentication information that identifies the Health Situation Responder 200 and can be used to validate the authenticity of the Registration Request;
b. A set of Health Situation types to which the Health Situation Responder 200 will potentially respond; and
c. Optionally one or more patient data conditions, where the Intervention Controller 210 is to validate that the patient data conditions are true for the patient to whom a Health Situation pertains and to refrain from reporting the Health Situation when this is not the case.

Control passes from step 310 to decision step 320, in which the Intervention Controller 210 determines whether or not to accept the registration request. In one embodiment, the determination depends on the Registration Request including valid authentication information, in accordance with the values specified in the Health Situation Responder Register 220. For example, the Health Situation Responder Register 220 includes for each Health Situation Responder entry a public key, the public key belonging to an asymmetric encryption key-pair, and the Registration Request includes a checksum encrypted using a private key known only to the Health Situation Responder. The Intervention Controller 210 decrypts the registration request using the public key in the Health Situation Register. If the decryption succeeds, the checksum is validated against the computed checksum of the registration request. In this example, the registration request is accepted if and only if both the decryption and the checksum validation succeed. It will be appreciated that many methods for authenticating such requests may be equally be practised without departing from the spirit and scope of the present disclosure.

Returning to step 320, if the Intervention Controller 210 does not accept the registration request, No, control passes to step 350, which rejects the registration request, and then control passes to an End step 350. Returning to step 320, if the Intervention Controller 210 accepts the registration request, Yes, control passes to step 340, which saves registration information associated with the requesting Health Situation Responder. In one implementation, the Intervention Controller stores a Health Situation Responder Profile for each registered Health Situation Responder, wherein the Health Situation Responder Profile includes a set of attributes for the respective Health Situation Responder. Such attributes may include, for example, but are not limited to, contact details, speciality, health care service provider number, registration identifier, and the like.

### Reference Personas

In order for a Health Situation Responder 200 to be successful at making medically appropriate Intervention Recommendations to patients, the Intervention Controller 210 will need to disclose to the Health Situation Responder 200 some information about those patients. In some embodiments, the Intervention Controller 210 only knows patient information associated with a patient during the time of a Health Situation. In alternative embodiments, the Intervention Controller 210 stores a profile for each patient and thus may recall historical information pertaining to a patient that has experienced a previous Health Situation.

According to one embodiment, patient information known to the Intervention Controller 210 about a typical patient includes values for one or more of the following attributes:
a. A patient identifier;
b. The patient's name;
c. The patient's address;
d. The patient's phone number(s);
e. The patient's date of birth;
f. The patient's medical conditions;
g. The medications, if any, currently being used by the patient; and
h. The medications, if any, the patient has used in the past.

In some embodiments, the Intervention Controller 210 does not disclose the patient information to the Health Situation Responder 200 directly, so as to ensure that the privacy rights and expectations of the patient are not violated. Instead, when reporting a Health Situation, the Intervention Controller 210 generates and discloses a Reference Persona to the Health Situation Responder 200. In this way, the embodiment ensures that the Health Situation Responder 200 receives sufficient information to make medically appropriate Intervention Recommendations, while not enabling the Health Situation Responder 200 to determine the identity of any of the patients for whom it makes the Intervention Recommendations.

For the purpose of the specification herein, the following terms are used in relation to attribute values that constitute the Patient Information known to the Intervention Controller 210 as specified below:
a. The term “Uniquely Identifying Value” is used to denote an attribute value that can be used to uniquely identify a particular patient in a patient population, e.g., a phone number;
b. The term “Partially Identifying Value” is used to denote an attribute value that can be used to identify a particular group of patients in a patient population where the group includes the patient the size of the group is smaller than a specified minimum, e.g., a street address; and
c. The term “Non-identifying Value” is used to denote an attribute value that is not either a Uniquely Identifying Value or a Partially Identifying Value.

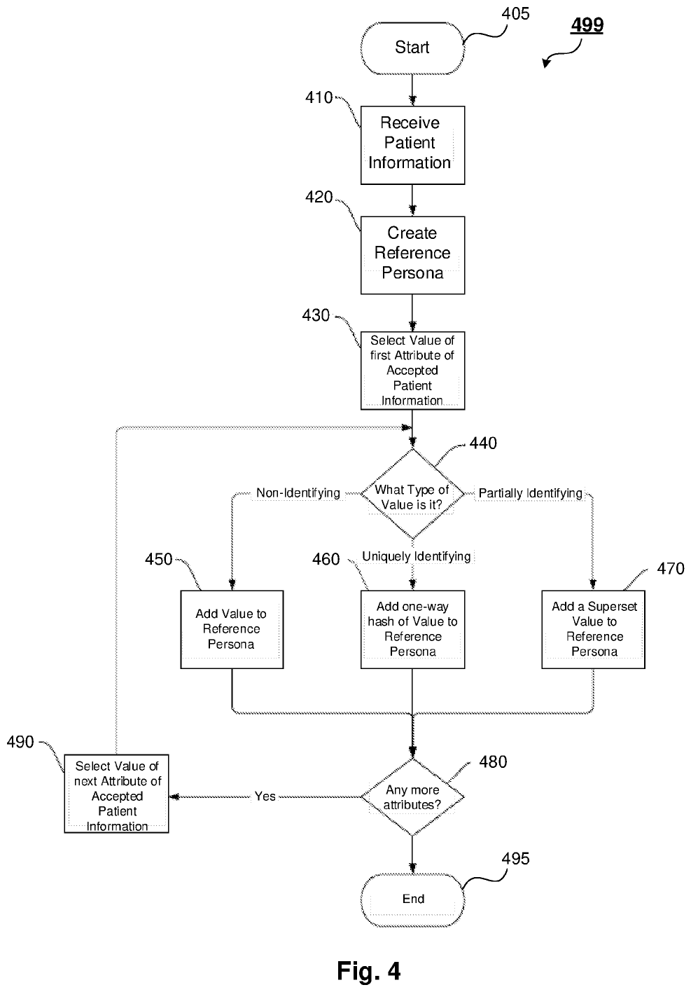

FIG. 4 shows, in simplified form, a flowchart 499 for the generation of a Reference Persona for a patient. The method begins at a Start step 405 and proceeds to step 410, which receives Patient Information. In one exemplary embodiment, this is done using a computer-implemented user interface when the patient registers as a user, whereby the patient uses a user interface displayed on a display of a computing device to enter a set of information. In one implementation, the user interface includes a set of one or more mandatory fields that must be completed by the patient in order to effect the registration.

Control passes from step 410 to step 420, which creates a Reference Persona data object for the registered patient. Control then passes to step 430, which selects a value of a first attribute of the received Patient Information. Control passes to decision step 440, which determines what type of value the selected value is. In particular, step 440 determines if the selected value is non-identifying, uniquely identifying or partially identifying.

In case the selected value is non-identifying, control passes from step 440 to step 450, which adds the selected value to the Reference Persona data object.

In case the selected value is uniquely identifying, control passes from step 440 to step 460, which generates a one-way cryptographic hash and adds that hash to the Reference Persona. It will be readily understood by a person skilled in the art that many different methods exist for generating such a hash value and that any suitable hashing method may be utilised to practise an embodiment of the present disclosure.

In case the selected value is partially identifying, control passes from step 440 to step 470, which chooses a superset value and adds the superset value to the Reference Persona. Whereas a partially identifying value is a value that is shared by a number of patients smaller than a pre-determined minimum size, the superset value is chosen to be shared by a larger number of patients in the patient population, wherein the larger number is larger than the pre-determined minimum size, such that the set of patients who share the superset value includes the set of patients who share the partially identifying value. In one exemplary embodiment, the date of birth attribute creates insufficiently large groups of patients, because too few patients share the same birthday. Many more patients are likely to share the same age than are likely to share the same birthday. In this embodiment, the superset value is the current age (in years) of the patient at the time the Reference Persona is generated. Step 470 includes adding the chosen Superset attribute value to the Reference Persona.

Control passes from each of steps 450, 460, 470 to decision step 480, which determines if there are any more attributes of the patient information that have not yet been processed in this way. If there are more attributes to be processed, Yes, control passes from step 480 to step 490, which selects the value of the next attribute of the received patient information and repeats the process. However, if at step 480 there are no more attributes of the patient information to be processed, No, control passes from step 480 to an End step 495 and the method 499 terminates.

### Interventions System

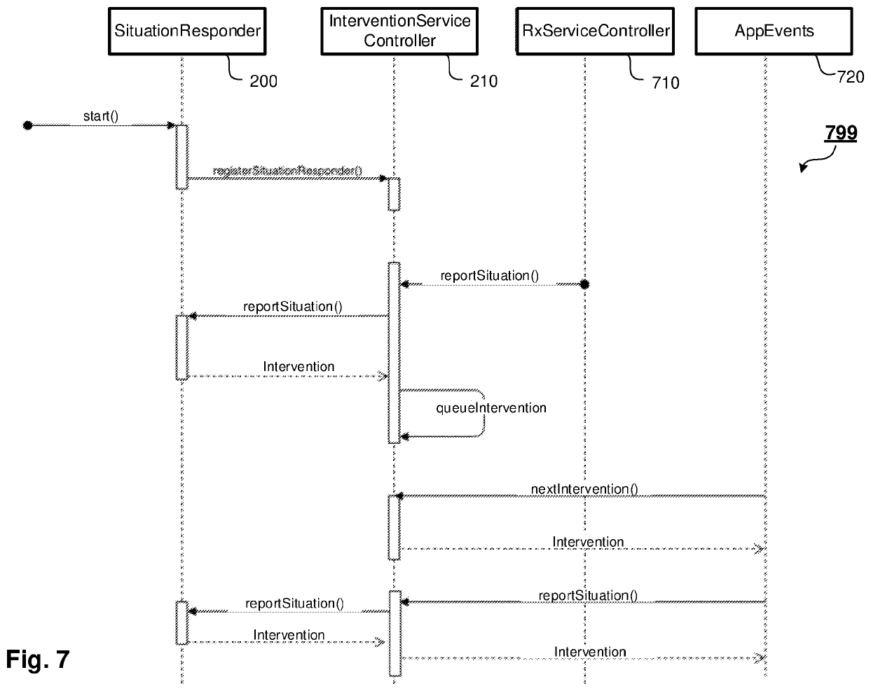

FIG. 7 depicts, in simplified form, a sequence diagram 799 illustrating some of the method invocations involved in the generation and processing of a Health Situation. A Situation Responder 200 sends a registerSituationResponder request to an Intervention Service Controller, corresponding to the Intervention Controller 210 of FIG. 2, in order to register that Situation Responder 200 with the Intervention Service Controller 210.

At a later point in time, a Service Controller 710, corresponding to the situation generator 240 of FIG. 2, reports an actionable situation to the Intervention Service Controller 210 by sending a reportSituation message. The Intervention Service Controller 210 receives the reportSituation message and then forwards the reportSituation message to the registered Situation Responder 200. The Situation Responder 200 replies to the Intervention Service Controller 210 with an Intervention Recommendation. The Intervention Service Controller 210 queues the Intervention Recommendation for action.

The example of FIG. 7 includes a polling mechanism, whereby an AppEvents module 720 periodically polls the Intervention Service Controller 210 for any other Intervention. Thus, once a predefined period of time has elapsed, the AppEvents module 720 sends a nextlntervention message to the Intervention Service Controller 210. On receipt of a nextlntervention message from an AppEvents module 720, the Intervention Service Controller 210 responds to the AppEvents module 720 by forwarding any Intervention Recommendation for action.

### System Implementation

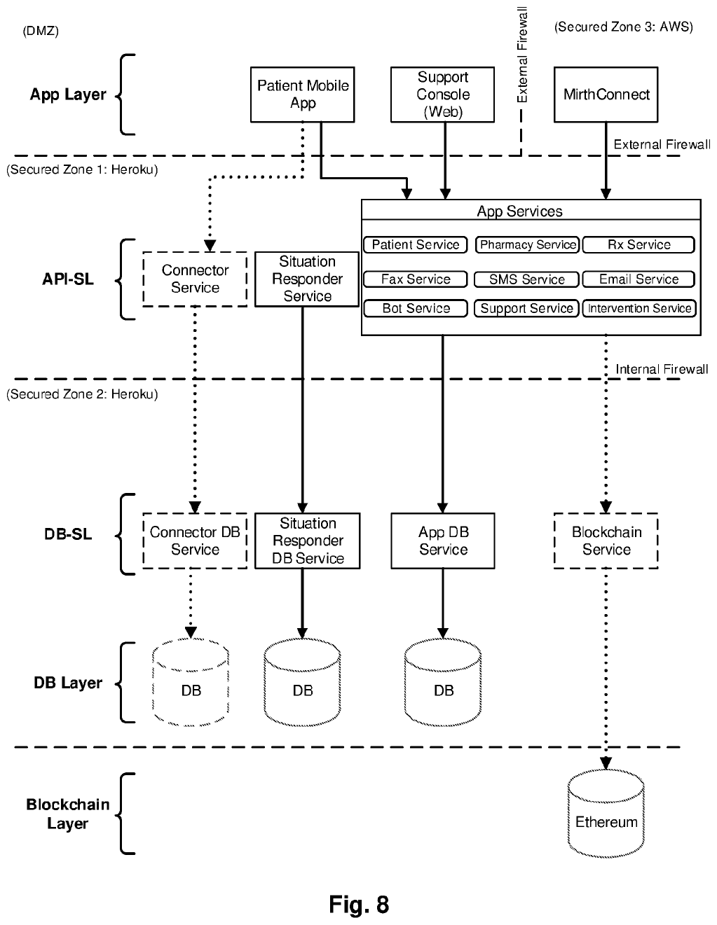

One embodiment provides a system for passively acquiring data from at least one data source. FIG. 8 is a schematic representation of functional modules for implementing the system, wherein the functional modules are grouped to the following physical layers and are shown with their respective interconnections:

| | |
|---|---|
| App Layer | Comprises applications that make use |
| | of backend services, typically running |
| | on client machines. |
| API-SL Layer | API Services Layer, comprising all |
| | internet-facing API endpoints. |
| | Components in this layer are all hosted |
| | using Heroku Private Shield Apps. |
| DB-SL Layer | Database Services Layer, comprising |
| | all database read/write services offered |
| | to API-SL components via API endpoints. |
| | Components in this layer are all hosted |
| | using Heroku Private Shield Internal |
| | Instances (not addressable from the internet) |
| DB Layer | Databases hosted in Heroku Postgres instances. |
| Blockchain | Ethereum blockchain |
| Layer | |

As can be seen from FIG. 8, the App Layer includes a Patient Mobile App, a Support Console and a MirthConnect module. The API-SL Layer includes a Connector Service, a Situation Responder Service, and App Services. The DB-SL Layer includes a Connector DB Service, a Situation Responder DB Service, an APP DB Service, and a Blockchain Service. The DB Layer includes a database corresponding to each of the modules in the DB-SL Layer, with the exception of the Blockchain Service. The Blockchain Service is connected to an Ethereum module in the Blockchain Layer, when the system is implemented using blockchain methodology.

The Patient Mobile App is a software application for execution on a computing device, such as a smartphone, accessed by a patient to interact with the system . The Patient Mobile App provides a user interface for patients to download, view, manage, and fill prescriptions. The Patient Mobile App also allows the presentation of various server-initiated interventions (value-added interactive services) to the patient, as a registered user of the system.

In one implementation, the Patient Mobile App is programmed using Expo (React Native) and JavaScript. In one implementation, security measures for the Patient Mobile App are implemented by utilising cached local data that is persisted using SecureStore for small data structures and encrypted files for large data structures. For example, files may be encrypted using the AES-JS Node Package Manager (npm) package.

The Support Console provides additional functionality to customer support personnel associated with administering the system. In one implementation, the Support Console is a web-based app that runs in a browser executing on a computing device, such as a personal computer. In order to protect data, the Support Console does not cache data in the browser and does not allow self-registration, with valid logins being pre-configured by a systems administrator. Further, the Support Console does not allow open browsing of data.

The App Services module bundles a set of micro-services so that those micro-services can run on a single Heroku app. These services provide the principal backend logic for importing and processing prescriptions, as well as providing all the API endpoints required by the Patient Mobile App for its operation. The App Services module may be programmed using Node.JS. In one implementation, the App Services are hosted using an Internet-facing Heroku Private Shield App.

The App DB Service provides database Object-relational Mapping (ORM) service to App Services, including automatic encryption and decryption of electronic Private Health Information (ePHI) to ensure that ePHI is never saved to the underlying database in unencrypted form.

The Situation Responder Service provides a service for automatically monitoring patient information and system events and recommending user-facing interventions to the Intervention Controller (in App Services), when appropriate. In one implementation, in order to receive patient/system event information from App Services, a System Responder must first register with App Services. App Services makes use of a predefined list of Situation Responders that are allowed to register. That list includes information used by App Services to authenticate the caller making the registration call using multiple factors, including public/private key encryption and IP address.

The Situation Responder DB Service provides database ORM services to Situation Responder Service, including automatic encryption and decryption of ePHI to ensure that ePHI is never saved to the underlying database in unencrypted form

The DB modules are databases for persisting system and user data.

MirthConnect is middleware for discovering (by regular polling) new prescriptions in a source EMR (electronic Medical Records System) and uploading these to App Services. In one implementation, MirthConnect is hosted using Amazon Web Services (AWS) Elastic Cloud Compute (EC2). In such an implementation, to maintain security no data is cached on an EC2 instance and a virtual private connection (VPN) is established to EMR.

The system may also include Auxiliary Services, which may be, for example, third party services including:
Freshdesk for end user support (App Services automatically publishes tickets to Freshdesk to recommend actions by administration support personnel);
Papertrail (event logging);
Twilio (SMS);
Amplitude (Metrics/Analytics); and
AWS S3 (File storage for some config files).

The Connector Service may be used by the Patient Mobile App to discover to which backend instance a patient should be connected, in the situation in which multiple system backend instances operate in production across multiple jurisdictions.

The Connector DB Service provides database ORM service to the Connector DB Service, including automatic encryption and decryption of ePHI to ensure that ePHI is never saved to the underlying database in unencrypted form.

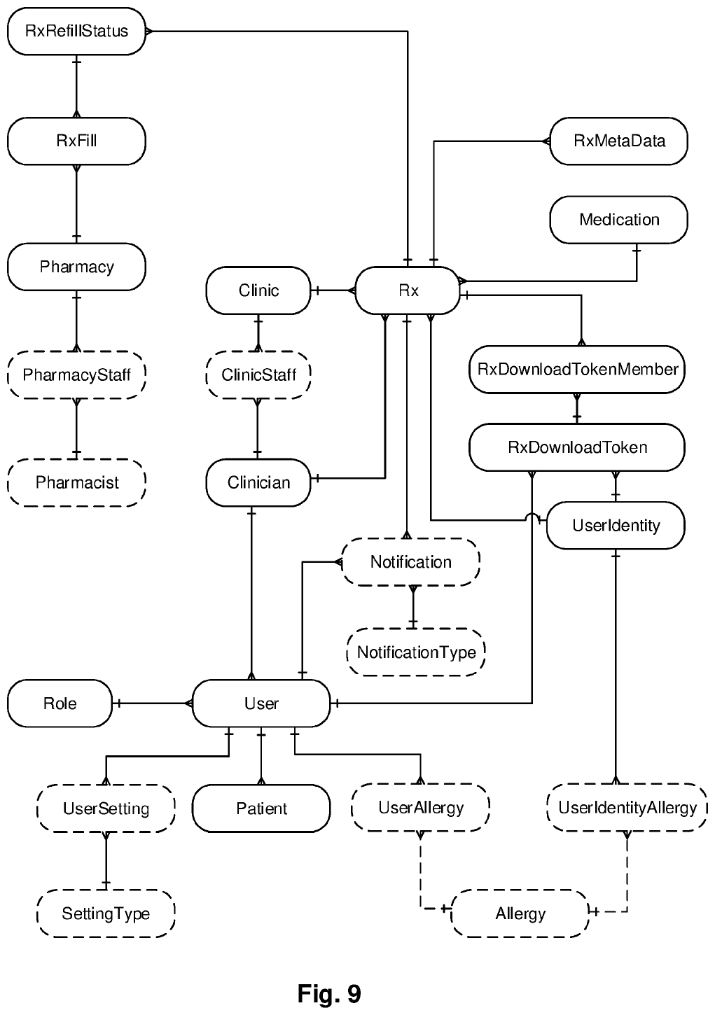

FIG. 9 is a schematic representation illustrating a sample set of principal data entities and various relationships, when a system in accordance with the present disclosure is implemented in relation to a healthcare environment. In particular, FIG. 9 shows relationships among a patient, a user, a clinic, a clinician, pharmacy, pharmacy staff, and pharmacist in an environment in which an intervention controller is utilised to control requests relating to health situations.

### INDUSTRIAL APPLICABILITY

The arrangements described are applicable to the computing and healthcare industries and particularly for the data acquisition and healthcare knowledge management industries.

The foregoing describes only some embodiments of the present invention, and modifications and/or changes can be made thereto without departing from the scope and spirit of the invention, the embodiments being illustrative and not restrictive.

Reference throughout this specification to “one embodiment,” “an embodiment,” “some embodiments,” or “embodiments” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

While some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practised without these specific details. In other instances, well-known methods, structures and techniques have not been shown in detail in order not to obscure an understanding of this description.

Note that when a method is described that includes several elements, e.g., several steps, no ordering of such elements, e.g., of such steps is implied, unless specifically stated.

In the context of this specification, the word “comprising” and its associated grammatical constructions mean “including principally but not necessarily solely” or “having” or “including”, and not “consisting only of”. Variations of the word “comprising”, such as “comprise” and “comprises” have correspondingly varied meanings.

Similarly, it is to be noticed that the term coupled should not be interpreted as being imitative to direct connections only. The terms “coupled” and “connected,” along with their derivatives, may be used. It should be understood that these terms are not intended as synonyms for each other, but may be. Thus, the scope of the expression “a device A coupled to a device B” should not be limited to devices or systems wherein an input or output of device A is directly connected to an output or input of device B. It means that there exists a path between device A and device B which may be a path including other devices or means in between. Furthermore, “coupled to” does not imply direction. Hence, the expression “a device A is coupled to a device B” may be synonymous with the expression “a device B is coupled to a device A”. “Coupled” may mean that two or more elements are either in direct physical or electrical contact, or that two or more elements are not in direct contact with each other but yet still co-operate or interact with each other.

As used throughout this specification, unless otherwise specified, the use of ordinal adjectives “first”, “second”, “third”, “fourth”, etc., to describe common or related objects, indicates that reference is being made to different instances of those common or related objects, and is not intended to imply that the objects so described must be provided or positioned in a given order or sequence, either temporally, spatially, in ranking, or in any other manner.

1. A system for delivering contextually relevant information, comprising:
a set of responders, each responder associated with at least one source of information;
a situation generator for parsing an agent data set received from an agent to identify an actionable situation; and
an intervention controller for:
sending a notification of said actionable situation to a selected set of said responders;
receiving an intervention recommendation from at least one responder, the intervention recommendation being contextually relevant information for the identified actionable situation;
determining whether the intervention recommendation is valid; and
forwarding the intervention recommendation to an intervention performer, when said intervention recommendation is valid.

2. The system according to claim 1, wherein determining whether the intervention recommendation is valid is based on past performance of the responder from which the intervention recommendation was received.

3. The system according to claim 1, wherein determining whether the intervention recommendation is valid is based on qualifications of the responder from which the intervention recommendation was received.

4. The system according to claim 1, wherein the selected set of responders is selected from the responders based on capabilities of the responders.

5. The system according to claim 1, wherein the agent data set is an electronic medical record (EMR) associated with a patient and the actionable situation is a health situation.

6. The system according to claim 1, wherein the agent is a system for handling EMRs.

7. The system according to claim 6, wherein the agent is selected from the group consisting of: a software application executing on a computing device, or a web-based application.

8. The system according to claim 1, wherein the actionable situation is a health situation based on at least one of a health condition, medical history, prescribed medication, and non-prescribed medication.

9. The system according to claim 1,
wherein the intervention performer is one of: a software application executing on a computing device, a web-based application, a chat bot, or a short message service (SMS) text message server.

10. The system according to claim 9, wherein the intervention performer is adapted to communicate with the agent.

11. The system according to claim 9, wherein the intervention performer is accessible by a person associated with the agent data set.

12. The system according to claim 11, wherein the agent data set is an EMR associated with a patient and the intervention performer is accessible by the patient, the intervention performer being adapted to display the intervention recommendation to the patient.

13. The system according to claim 1, wherein each responder in the set of responders is registered in a health situation responder register.

14. A method of passively acquiring knowledge distributed among at least one source of information, comprising the steps of:
receiving at an intervention controller an agent data set associated with an agent;
analysing, by a situation generator associated with the intervention controller, the agent data set to identify an actionable situation;
notifying, by the intervention controller, the actionable situation to a set of at least one responder, wherein each responder has access to at least one source of information relating to the actionable situation;
determining, by at least one of said responders, an intervention recommendation based on the actionable situation;
sending, by said at least one of said responders, said intervention recommendation to said intervention controller;
forwarding, by said intervention controller, said intervention recommendation to an intervention performer.

15. The method of claim 14, wherein said agent data set is an electronic medical record (EMR) containing patient information.

16. The method according to claim 14, comprising the further step of:
determining, by the intervention controller, whether to act upon the intervention recommendation.

17. The method according to claim 14, wherein:
said agent data set is an EMR relating to a patient;
said actionable situation is a health situation;
said responder is a health situation responder; and
said intervention recommendation is based on patient information in said EMR, said patient information relating to at least one of: a health condition experienced by the patient, medical history of the patient, and medicine being taken by the patient or prescribed to be taken by the patient.

18. The method according to claim 14, wherein each of said at least one responder is registered with said intervention controller.

19. The method according to claim 14, wherein said intervention performer is one of: a software application executing on a computing device, a web-based application, a chat bot, or a short message service (SMS) text message server.

20. The method according to claim 14, wherein said intervention controller is implemented using a computing device connected to a communications network and is adapted to communicate via said communications network with each of said responders, said situation generator, and said intervention performer.
